(19) United States
(12) Patent Application Publication
(10) Pub. No.: US 2016/0214952 A1
(43) Pub. Date: Jul. 28, 2016

(54) IMPROVED PROCESS FOR MANUFACTURE OF TETRAHYDROFURAN

(71) Applicant: INVISTA TECHNOLOGIES S.a r.l., St. Gallen (CH)

(72) Inventors: Graham R. AIRD, Darlington (GB); Clive Alexander HAMILTON, Swainby (GB); Lauren Emily Sheridan BECK, Fife (GB)

(73) Assignee: INVISTA NORTH AMERICA S.A R.L., WILMINGTON, DE (US)

(21) Appl. No.: 14/917,362

(22) PCT Filed: Sep. 9, 2014
(86) PCT No.: PCT/US2014/054842

§ 371 Date: Mar. 8, 2016

Related U.S. Application Data
(60) Provisional application No. 61/875,828, filed on Sep. 10, 2013.

(51) Int. Cl. C07D 307/08

(52) U.S. Cl. CPC C07D 307/08

(57) ABSTRACT

The disclosed process relates to an improved process for manufacturing THF from a reaction mixture comprising BDO in the presence of an acid catalyst in a reaction vessel comprising a distillation reaction zone, wherein the acid catalyst is suspended in a vapor-rich region in the distillation reaction zone.

### CROSS-REFERENCE TO RELATED APPLICATIONS

This application claims the benefit of U.S. provisional application No. 61/875,828, filed Sep. 10, 2013, the disclosures of which are incorporated herein by reference in their entireties.

### FIELD OF THE INVENTION

The disclosed process relates to an improved process for manufacturing tetrahydrofuran (“THF”) from a reaction mixture comprising 1,4-butanediol (“BDO”) in the presence of an acid catalyst.

### BACKGROUND OF THE INVENTION

In current processes for manufacturing THF from a reaction mixture comprising BDO in the presence of an acid catalyst, for example, sulphuric acid, in a distillation reaction zone, the acid catalyst is in the reaction mixture to accomplish dehydration of the BDO and ring closure. This process necessitates the use of high grade materials to avoid corrosion of the process vessels due to the strong acidity of the hot process liquor. Further, this process generates an accumulating high concentration of an acidic black viscous tar which represents a yield loss and operational problems. Literature teaches that the same chemistry can be carried out with a solid acid catalyst (see Vaidya et al, Applied Catalysis A: General 242 (2003), 321-328). In the latter case the reaction takes place on the surface of the catalyst rendering the mixture pH neutral. However, in a continuous process, even with the use of a solid acid, catalyst, periodic costly maintenance for regeneration of catalyst with significant surface tar is required.

U.S. Patent Application Publication No. 2008/0161585A1 and its European counterpart EP1939190B1 disclose use of ZrSO₄ as catalyst in either a liquid or gas phase process to manufacture THF from butanediol. The publication drawing shows a reactor linked to a distillation column. THF and water are removed from the top of the distillation column while butanediol from the base of the column is recycled to the reactor.

U.S. Pat. No. 5,099,039A (“the '039 patent”) relates to a process for the production of THF from BDO wherein polybenzimidazole (“PBI”) catalyzes the conversion of BDO to THF. The PBI catalyst is in the protonated or acidic form as described in example I of the '039 patent.

Japan Patent No. 07118253A discloses only a particular catalyst as a means for effecting the conversion of BDO to THF. It does not mention tars or byproducts.

China Patent No. 101298444B discloses using a strongly acidic ion exchange resin to convert BDO to THF at an operating temperature of up to 120° C. It does not mention suspending the acidic ion exchange resin above boiling BDO in a distillation reaction zone.

Chemical Engineering Science, 56, 2001, 2171-2178, discloses conversion of BDO to THF in a batch stirred pot reactor using an ion exchange resin contained in a basket. The basket is initially suspended in the pot until the batch reactor is heated. At that point, the basket is dropped into the pot, i.e., the catalyst is in the reaction mixture in the batch stirred pot reactor. Boiling vapor is not used to effect the reaction, which is done in the liquid phase under pressure to prevent evaporation of product.

In light of current practices and the disclosures of art, a simple economical process is needed for manufacturing THF from a reaction mixture comprising BDO in the presence of an acid catalyst to avoid these issues.

### SUMMARY OF THE INVENTION

The present invention provides an economical improved process for manufacturing THF from a reaction mixture comprising BDO in the presence of an acid catalyst at reaction conditions.

One aspect of the disclosed process is directed to an improved process for making tetrahydrofuran, the process comprising:

providing a reaction mixture comprising 1,4-butanediol to a first zone;

maintaining conditions of temperature and pressure in the first zone sufficient to produce a vapor-rich region comprising the reaction mixture;

providing a second zone in the vapor-rich region between the first zone and a vapor condenser, wherein the second zone comprises an acid catalyst;

maintaining conditions of temperature and pressure in the second zone sufficient to effect dehydration of 1,4-butanediol and ring closure to form a product comprising tetrahydrofuran; and

recovering the product.

### BRIEF DESCRIPTION OF THE FIGURES

FIG. 1 is a representation of process according to one embodiment in Example 4.

FIG. 2 is a representation of process according to another embodiment in Example 5.

FIG. 3 is a representation of a process equipment arrangement used according to Examples 1 and 2.

FIG. 4 is a representation of a process equipment arrangement modified according to Example 3.

### DETAILED DESCRIPTION OF THE INVENTION

One aspect of the disclosed process is directed to an improved process for making tetrahydrofuran, the process comprising:

providing a reaction mixture comprising 1,4-butanediol to a first zone;

maintaining conditions of temperature and pressure in the first zone sufficient to produce a vapor-rich region comprising the reaction mixture;

providing a second zone in the vapor-rich region between the first zone and a vapor condenser, wherein the second zone comprises an acid catalyst;

maintaining conditions of temperature and pressure in the second zone sufficient to effect dehydration of 1,4-butanediol and ring closure to form a product comprising tetrahydrofuran; and

recovering the product.

As a result of intense research, we have found that we can economically and effectively manufacture THF from a reaction mixture comprising BDO. In certain embodiments, the improved process can be in the presence of a solid catalyst in a reaction vessel comprising a distillation column reaction zone at distillation reaction conditions. The disclosed process avoids the problems associated with current practices of acid catalyzing the dehydration of BDO in a distillation reaction zone to produce product comprising THF. The improvement of the present invention allows lower cost materials of construction for reaction zone equipment with significant reduction in tar formation. Reduction in tar formation represents a significant yield improvement along with much less frequent equipment down-times and shutdowns for cleanup and maintenance.

Examples of the reaction vessels, include, but are not limited to, a continuously stirred tank reactor, a plug flow reactor, or a trickle flow reactor. In one embodiment, the reaction vessel is a continuously stirred tank reactor. In a further embodiment, the reactor vessel is a tank with external circulation through a pump. In another embodiment, the reaction vessel is a plug flow reactor. In yet another embodiment, the reaction vessel is a trickle flow reactor with the liquid flowing down and the vapors flowing up.

The term “BDO” as used herein represents 1,4-butanediol, also known as 1,4-butylene glycol, having the formula HOCH₂CH₂CH₂CH₂OH.

The phrase “reaction mixture comprising BDO” is intended to refer to a mixture primarily containing 1,4-butanediol, but may also contain minor amounts of impurities such as, for example, 2,4-hydroxybutoxy tetrahydrofuran; 2-methyl-1,4-butanediol and 2-butene-1,4-diol. In one embodiment, the reaction mixture comprises about 40-100 wt % BDO, 0-30 wt % THF and 0-30 wt % water. In another embodiment, the reaction mixture comprises about 50-100 wt % BDO, 0-25 wt % THF and 0-25 wt % water. In yet another embodiment, the reaction mixture comprises about 60-100 wt % BDO, 0-20 wt % THF and 0-20 wt % water. In a further embodiment, the reaction mixture comprises about 70-100 wt % BDO, 0-15 wt % THF and 0-15 wt % water. In yet another embodiment, the reaction mixture comprises about 80-100 wt % BDO, 0-10 wt % THF and 0-10 wt % water.

In some embodiments, the reaction mixture comprising BDO is a liquid mixture that is used to generate the vaporized feed to the reaction zone.

The term “THF” as used herein represents tetrahydrofuran, also known as cyclotetramethylene oxide, represented by the formula C₄H₈O. Percentages used herein are in weight % unless otherwise indicated.

The term “yield loss” as used herein means a molecular loss of a useful reactant via its conversion to form undesired reaction by-product(s). The yield loss of a chemical reactant is due to inefficiencies in the chemical conversion process. A zero-percent yield loss means no chemical reactant has been lost to undesired chemical by-products. A 100-percent yield loss means all chemical reactant has been lost to undesired by-products. Specifically, the yield loss of BDO to the byproduct tar formation means the molecules of BDO that are associated with the undesired tar formation. A lower yield loss is desirable in any chemical reaction system.

The term “tar” as used herein represents mass accumulation of side reaction products from undesirable chemical interactions involving the product, catalyst and/or starting feed at the reaction conditions. Tar formation and build-up is associated with the loss of desired product selectivity and yield, and is also responsible for coloring of the reaction mixture. Tar formation has detrimental effects on the equipment operability and performance. Increasing levels of tar is undesirable from any reaction viewpoint.

In some embodiments, the yield loss of butanediol to tar is less than 5.0, or less than 4.0, or less than 3.0 percent by weight. In other embodiments, the yield loss of butanediol to tar is less than 2.0 percent by weight. In a further embodiment, the yield loss of butanediol to tar is less than 1.0 percent by weight.

In some embodiments, the reaction mixture is characterized by a Hunter Colorimetry index, L*/a*/b*, and the Hunter Color index, L* is from about L*=0 to about L*=100, a* is from about a*=−100 to about a*=100 and b* is from about b*=−100 to about b*=100.

In some embodiments, the tar formation is determined by the mass totaling method.

The main purpose of the first zone is to generate vapor feed for the reaction without over-heating the reaction mixture. In some embodiments, the first zone may be a liquid-vapor heat exchanger with the heat-transfer surface that adequately heats the liquid reaction mixture to its bubble-point temperature. In other embodiments, the first zone is a reboiler. The reboiler may be of any of such conventional types as, but not limited to, shell-and-tube, calendria, thermosyphon, film evaporator, jacketed and/or heating coiled kettle, forced-circulation, fuel-fired. The reboiler may be equipped with a liquid circulation pump and a draw-off point for removing the liquid to maintain the steady state. Steam is normally used as heat source, however, other heat transfer fluids as hot-oil and DowTherm™ may be used depending on the temperature range. In fuel-fired type, fuel oil or fuel gas such as natural gas or LPG may be used. The reboiler may be installed for an efficient vapor disengagement from the liquid while maintaining a good contact between the heat transfer surface and liquid.

In some embodiments, the second zone comprises a solid catalyst section that accomplishes the desired chemical conversion in the catalyst presence. In some embodiments, the second zone may comprise of a catalytic reaction section integrated with the distillative separation stages. In other embodiments, the catalytic reaction section may be external to the distillative separation stages. The catalytic reaction section affords a chemical conversion reaction in the presence of liquid and/or vapor phases. In further embodiments, the solid catalyst section is located in the vapor-rich environment of the second zone.

In some embodiments, the second zone further comprises an annular chimney reactor. In other embodiments, the second zone is externally connected with the solid catalyst section. In one embodiment, the second zone comprises the catalyst section internally.

In some embodiments, when steady state is reached in the disclosed process, the product comprises 80±20/20±20, weight/weight, THF/water.

In some embodiments, the vapor condenser accepts the overhead vapor stream from the distillative reaction vessel and condenses into a liquid product. The vapor condenser may be vapor-liquid heat exchanger with the heat-transfer surface that is adequate to cool the overhead vapors to, or sub-cool below, its dew point temperature. The vapor condenser is oriented such that the condensed liquid flows out of the unit without letting the uncondensed vapors escape. In a conventional arrangement, the vapor condenser may be equipped with a liquid seal and a condensed liquid accumulator to prevent any vapor escape. The condenser may be equipped with a condensate pump-around and a draw-off point to establish steady state while in operation. Conventional vapor-liquid condensers may be of the surface-type such as shell-and-tube, cross-flow, or contact-type such as direct contact and spray condenser.

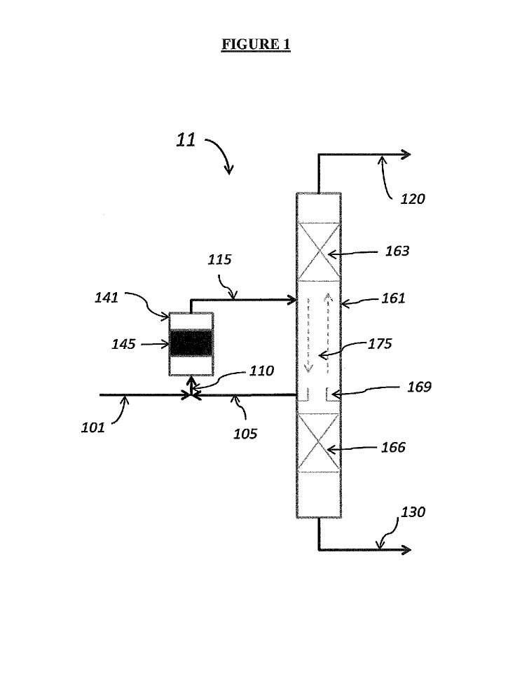

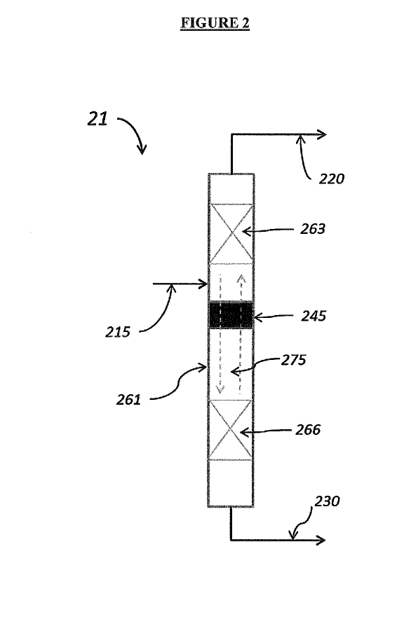

In some embodiments, the acid catalyst section may be located external to the second zone, e.g., a distillation column (see FIG. 1) or contained inside the second zone, e.g., a distillation column packing bed (see FIG. 2). The reaction may be conducted under high pressure conditions in which it remains in the liquid phase or it may be conducted at sufficiently low pressure that the THF boils out. The reactor vessel may be designed to operate adiabatically, or designed to be heated to control temperature directly.

In some embodiments, the conditions in the second zone are such that the BDO in the reaction mixture reacts to form THF, and include a temperature of from 80 to 250° C. and pressure from 200 to 10,000 mbar absolute. Examples of such reaction conditions include a temperature of from 110 to 250° C., such as from 120 to 150° C., and pressure from 950 to 8,000 mbar absolute. Heat may be applied to the reaction zone directly to control temperature conditions therein. The contents may vaporize in the reactor upon formation of THF and water, or it may be kept in the liquid phase if the pressure is kept high enough within the above ranges.

In some embodiments, the acid catalyst can be in the forms of solid, semi-solid, and/or of gel consistency. In one embodiment, the acid catalyst is a solid catalyst. In other embodiments, the solid catalyst is a strongly acidic ion exchange resin. In other embodiments, the solid catalyst can be mineral-based supported acid catalysts, such as zeolites. In one embodiment, the solid resin catalyst can be selected from commercially available strongly acidic, cationic polymeric catalysts. Non-limiting examples of such solid acid resin catalyst include Amberlyst™ 35, Amberlyst™ 70, Puralite™ CT and combinations thereof. In some embodiments, the suitable solid acid resin catalyst has an acid equivalency of at least 1, for example from at least 1 to 10, such as from 3 to 10.

In some embodiments, the product separation and recovery may be accomplished by suitable process techniques, such as, appropriate distillation means.

### Overview of FIG. 1

A more detailed description of a representative process for THF production from BDO is shown as arrangement 11 in FIG. 1, which provides a simplified schematic representation of such a process. FIG. 1 shows a distillative reaction column 161, equipped with an overhead condenser (not shown) to condense a distillate vapor steam 120 into a distilled liquid product, a bottoms reboiler (not shown) to assist boilup of the reaction liquid stream 130, a liquid sidedraw collection section 169, and optionally, distillative separation stages via sections 163 and 166. Sections 163 and 166 may include theoretical stages in the form of structured, random packings or distillative trays that are commonly used in conventional vapor-liquid distillation. The desired vapor-liquid traffic, shown via 175, may be achieved by adjusting reboiler boil-up rate and condenser refluxing. Those skilled in the art of distillation will know how to achieve balanced column hydraulics without stage drying due to excessive vapor up-flow or flooding due to excessive liquid down-flow in the column.

According to a non-limiting embodiment shown in FIG. 1, a catalyst section 141 may be provided external to the column 161, which houses an appropriate bed of solid catalyst 145. A fresh reaction feed comprising BDO may be introduced via stream 101. Stream 101 may be sub-cooled liquid, saturated liquid, partial mixture of vapor and liquid or saturated vapor. The fresh feed stream 101 is mixed with the liquid side-draw stream 105 from the liquid collection section 169 of the column 161, and the combined stream 110 is fed to the catalyst section 145. A provision may be made to control the temperature of the mixed feed stream 110 at the inlet of the catalyst section 141.

The reacted effluent vapor stream 115, from the catalyst section 145, comprising unconverted BDO, THF product, water, and optionally, other byproducts along with any residual tar, is taken back to the column 161 via appropriate piping and vapor feed port. The temperature and pressure conditions across the column 161 are maintained such that the THF product along with water are distilled in the overhead via stream 120. The unconverted BDO and other high-boiling components such as tars flow down as equilibrated liquid. The tars and BDO are separated in the section 166 and tars are concentrated as stream 130. The column 161 therefore provides the necessary separation between the low-boiling THF product, higher-boiling reactant BDO and highest-boiling tars.

Stream 120 comprises of the THF product and water that are formed in the catalyst section 145. Further separation and refining for yielding purified THF is accomplished by processing stream 120 via known methods for THF purification, including but not limited to, conventional distillation, azeotropic distillation, pressure distillation, membrane separation, dehydration beds, etc.

The bottoms stream 130 essentially comprises unconverted BDO and other by-products including tars and with negligible THF. Stream 130 is taken to the reboiler and heated to provide the BDO-rich vapor feed (not shown) to the column 161.

At steady state, the fresh feed stream 101 flow rate is roughly matched with the net distilled liquid take-off (not shown) from the overhead, which may be the entire stream 120 or a portion of stream 120. In the case of stream 120 portion taken as overhead product, the remaining portion may be fed back to the column 161 as reflux to balance the column hydraulics. Several purge points may be selected at appropriate locations to control and prevent the byproducts from building up in the process.

### Overview of FIG. 2

In FIG. 2, arrangement 21 shows a non-limiting embodiment of the disclosed process for the THF production from BDO. A distillative reaction column 261 is equipped with an overhead condenser (not shown), bottoms reboiler (not shown), a column feed entry point 215 at one or multiple axial locations, and one or multiple theoretical separation stages via sections 263 and 266. Additionally, a catalyst section 245 is mounted internally between the overhead condenser and bottoms reboiler. The vapor-liquid traffic passes through the catalyst section, shown as 275. The fresh column feed via stream 215 may be fed either above, middle or below the catalyst section 245.

At steady state, the fresh column feed 215 rate is roughly matched with the net overhead product draw (not shown). The overhead product draw may be the entire stream 220 or a portion thereof. The bottoms BDO-rich stream 230 is taken to the reboiler where the boilup heat is provided and the BDO-rich vapors (not shown) are fed back to the column 261.

The internal catalyst section 245 may be one or multiple sections, arranged to fit inside the column internals via proper bracketing and supporting. These sections may be packed with proper voidages to provide acceptable pressure drop across the column. The catalyst section may be a suitable catalytic structured packing type, rolled, corrugated sheet sections, reticulated foam type or wash-coated catalytic substrates such as honeycomb or monolithic structures.

In arrangements 11 and 21, adequate temperature and pressure measurement points may be provided to obtain the desired reaction and distillative environment. Conventionally, the bottoms reboiler will be equipped with over-temperature controls and liquid level controls to minimize over-heating and drying of the heated surface. The overhead condenser will be equipped with proper coolant feed controls and liquid level controls for efficient vapor condensing. The catalyst section may operate either adiabatically, isothermally or with controlled temperature profile via the available means.

The following Examples demonstrate the present invention and its capability for use. The invention is capable of other and different embodiments, and its several details are capable of modifications in various apparent respects, without departing from the scope and spirit of the present invention. Accordingly, the Examples are to be regarded as illustrative in nature and not as restrictive. All percentages are by weight unless otherwise indicated.

### Determination of Tar Formation in Reaction Mixture

Hunter Colorimetry Method—

In the examples, color measurements are determined as described in Hunter Lab Applications Note, Vol. 8, No. 9, 2008, 1-3, and the association of color with tar formation is made with a Hunter Lab color measurement instrument. The reaction mixture in the process is characterized by a Hunter Color index, L*/a*/b*, wherein, the Hunter Color index, L* is from about L*=0 to about L*=100, a* is from about a*=−100 to about a*=100 and b* is from about b*=−100 to about b*=100. A convenient key to these color measurements is that higher L* and lower b* values indicate lower tar content. As an illustration, an L* value of “0” indicates totally opaque/black coloration due to excessive tar; and an L* value of 100 indicates completely clear of the tar formation.

Mass Totaling Method—

In the following examples, a quantitative determination of tar formation is made by analyzing the reaction liquid mixture at steady-state conditions, measuring composition of known components in the reaction mixture, and by mass subtraction of the combined weight percentages of the known components from 100%.

### EXAMPLES

As used in the Examples, the term “BDO” refers to 1,4-Butanediol (1,4-BDO; Chemical Abstracts Registry Number CAS No. 110-63-4). Table 1 (1,4-BDO by INVISTA S.à.r.l.) gives a typical composition of the BDO used.

TABLE 1

| Specification | | Physical Property Data | |
|---|---|---|---|
| Colour, APHA | <10 | Molecular weight | 90.1 |
| Water wt. % | <0.05 | Hydroxyl number+ | 1240-1246 |
| Carbonyl No., | <0.1 | Viscosity, cP (mPa. · s), | 70-73/37-40 |
| mg KOH/g | | 25°/40° C. | |
| Assay, wt. % | >99.5 | Density, 40° C. g/mL (Mg/m3) | 1.014 |
| | | Melting point++, ° C. | 19-20 |
| | | Boiling point, ° C. | 228 |

+calculated
++Freezing point

The concentrated H₂SO₄ used has a typical composition of 98% by weight.

The Amberlyst™ 35 used is obtained from Dow Chemicals. The Amberlyst™ 35 W resin form is washed with demineralized water and dried in an oven overnight at 90° C.

### Example 1

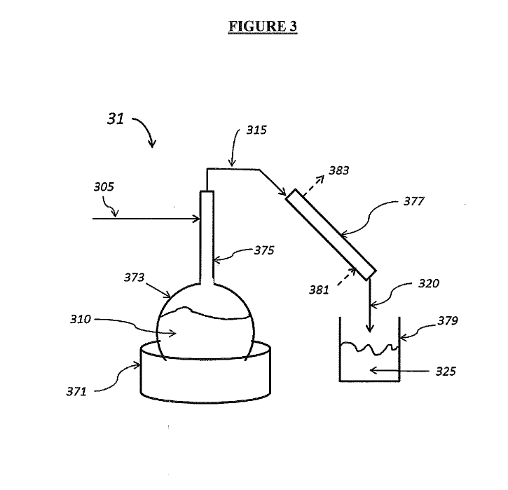

FIG. 3 shows the equipment arrangement 31 as used in this example. A 250-mL round-bottom glass reaction flask 373 is fitted with a long distillative neck 375 that has provision for liquid feed addition via stream 305. The other two necks are used to install a thermocouple and discharge line. An electric round-bottom flask isomantle 371 is used to provide heat to the reaction mixture 310. A water-cooled condenser 377 is connected to condense the vapors generated via stream 315. The condenser 377 is connected to a cooling fluid supply and cooling fluid return line via streams 381 and 383, respectively. The condensed distillate stream 320 is collected in a distillate receiver 379. Once a steady-state is reached, periodic samples of the distillate liquid 325 are taken from the distillate receiver 379 and analyzed via Gas Chromatography for organics and Karl-Fischer analysis for water. Also, periodic samples of the reaction mixture 310 are taken for the composition, color and tar measurements.

In this example, 1.25 grams of H₂SO₄ is added to 98.75 grams of BDO, i.e., a 1.25% H₂SO₄ in BDO solution, and charged to the reaction flask 373. Heat is applied via isomantle 371. When the reaction mixture began to boil (˜130° C.) and THF/water collection commenced in the distillate receiver 379, additional BDO is added continuously via stream 305 to maintain liquid level in the reaction flask 373. The steady state flow rate of BDO achieved is about 3.5 mL/minute with the bulk, reaction liquid temperature reaching about 143° C. This produces about 3.5 mL/minute of an 80:20, wt/wt mixture of THF and water distilled overhead via stream 315, and eventually, the condensed liquid stream 320.

The experiment is continued for over 60 hours during which time tar formation is visually observed in the reaction flask 373 as severe discoloration, i.e., the bulk reaction mixture 310 turns black and opaque after 6 hours. The liquid phase samples of 310 taken from the reaction flask 373 has a steady state composition of 75% BDO, 3% THF and 5% water, giving about 15% tar by the mass totaling method. The color of 310 by Hunter Colorimetry method is determined to be steady state L*/a*/b* values of 25/17/40 following a ten-fold dilution of the samples in BDO.

### Example 2

Using the equipment arrangement and procedures described above and in FIG. 3, Example 1 is repeated with 5.0 grams of solid acid resin catalyst, i.e., Amberlyst™ 35, replacing the H₂SO₄, and 95.0 grams of BDO. This reaction mixture is charged into the reaction flask 373. The acid equivalence of dried Amberlyst™ 35 is 25% of that of H₂SO₄; hence the 5.0 grams added has the same acid equivalence as 1.25 grams of H₂SO₄ as used in Example 1. The reaction mixture heat-up, distillation vapor condensation and distilled liquid collection procedures described in Example 1 are followed herein. Very similar results are obtained in terms of reaction liquid phase (i.e., sample of 310 in FIG. 3) composition and color, specifically 80% BDO, 5% water and 3% THF, giving about 12% tar by the mass totaling method, and an L*/a*/b* of 62/13/80 by Hunter Colorimetry method. The distilled phase composition is also very similar at ˜80% THF and ˜20% water (i.e., sample of 325 in FIG. 3). The steady state feed rate of BDO (stream 305 in FIG. 3) is a little higher than that of Example 1 at about 4.0 ml/minute, and is considered within experimental error.

### Example 3

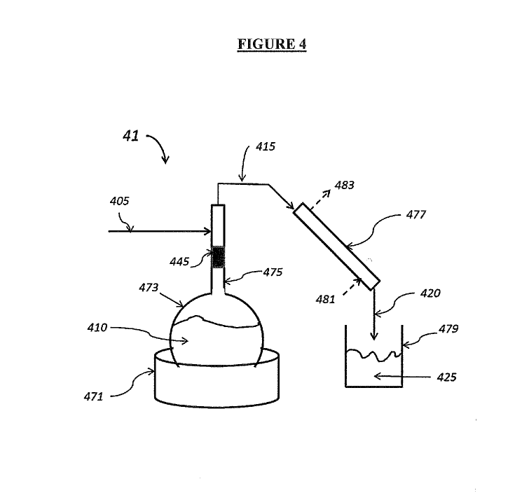

The original equipment arrangement described in Example 1 and shown in FIG. 3 is modified to include a solid catalyst section in the distillative neck. The new arrangement 41, as used in this example, is shown in FIG. 4. About 1.0 gram of solid acid resin catalyst, i.e., Amberlyst™ 35, is contained within a stainless steel mesh container and suspended in the distillative neck 475. The mesh container is placed in a 4.2 mL glass cup suspended above the reaction flask 473 containing 100 ml of BDO. The suspended mesh container and glass cup containing the solid acid catalyst resin is referred to as the “internal reactor” section 445 in FIG. 4. Heat is applied to the round-bottom reaction flask 473 via isomantle 471 and once the liquid started to boil, additional BDO is added continuously via stream 405 through the suspended internal reactor. The additional BDO flow rate is adjusted to match with the THF/H₂O flow distilling over the top via stream 415, and eventually, the condensed liquid stream 420. At that point there is no accumulation of BDO in the round-bottomed flask 473 and the process is at steady state, indicated by the constant bulk reaction liquid level in 473. At steady state, about 2 mL/minute of BDO (stream 405) is converted to about 2 mL/minute of THF/H₂O (stream 420) with this process configuration. The internal reactor 445 temperature is about 135° C. with the boiling BDO (i.e., in bulk liquid 410) from the round-bottomed flask 473 serving as the vaporic heat source. The round-bottomed flask 473 has a steady state temperature/boiling point of about 215° C. and a composition of 93% BDO, 5% THF and 2% water, giving only 1% for tars by the mass totaling method. Color of the samples 410 material is measured to be L*/a*/b* of 96/−1.6/16 by Hunter Colorimetry method. The surprising reduction in tar content is apparent from the significantly higher L* color along with lower b* color compared to the values from Examples 1 and 2, and the much higher liquid phase BDO content in the bulk liquid 410. The distilled phase composition is measured to be approximately 80% THF and 20% water (i.e., sample of 425 in FIG. 4).

### Example 4

Referring to FIG. 1, an industrial-scale, continuous distillation column 161 is equipped with an industrial reboiler (not shown) at the base and industrial vapor condenser (not shown) in the overhead. The column shell is made up of carbon or stainless steel while all column internals are stainless steel. In FIG. 1, section 163 and/or section 166 of the column 161 comprise of theoretical separation stages in the form of structured, random packings or distillative trays that are commonly used in liquid-vapor distillation. The column hydraulics is set by the bottoms reboiler with adequate boil-up rate to provide the necessary vapor up-flow traffic through the column and the overhead condenser which provides sufficient liquid down-flow through the reflux rate. The effective vapor and liquid flow traffic is shown by 175 which keeps the column from either drying or flooding. Those skilled in the art will know how to adjust the reboiler boil-up and condenser reflux for achieving stable column hydraulics.

A stationary bed 145, containing solid acidic resin catalyst pellets, is mounted in an external reactor 141. A liquid stream 105 is drawn from the column via tray 169 to reactor 141, while reactor effluent consisting of vapor and optionally liquid is returned to column 161 via line 115. There are more than one theoretical separation stages between the reactor effluent return line 115 and overhead condenser and more than one theoretical stages between the liquid draw-off stream 105 and bottoms reboiler.

A fresh reaction feed comprising BDO is introduced via stream 101 to replenish the converted BDO in the system. The fresh feed stream 101 is mixed with the liquid side-draw stream 105 from the liquid collection section 169 of the column 161, and the combined stream 110 is fed to the catalyst section 145. A provision is made to monitor and control the temperature of the combined feed stream 110 at the inlet of the reactor 141.

The catalyst reaction zone 145 conditions are maintained such that the reaction of BDO to THF proceeds with high conversion and desired product selectivity. As the reaction continues the by-product water and THF product are driven out of the reaction zone as vapor. The vapor mixture (i.e., stream 120) is condensed in the overhead condenser and recovered as 80120:20120% THF:water distillate mixture. The THF-rich liquid distillate is drawn out of the system and processed downstream.

Fresh BDO is charged via stream 101 at a steady feed rate to replenish the converted feed. At steady state, the overhead product mass flow rate is roughly matched with the fresh BDO addition. The column base continues to boil BDO which provides the reactant feed to the reaction zone 145.

The undesired tar byproduct is concentrated and removed in the column base where the maximum temperature is observed. The bottom liquid stream 130 composition comprises mainly BDO with trace amounts of THF, water and a few % by weight tars.

### Example 5

Referring to FIG. 2, an industrial-scale, continuous distillation column 261 is equipped with an industrial reboiler (not shown) at the base and industrial vapor condenser (not shown) in the overhead. The column shell is made up of carbon or stainless steel while all column internals are stainless steel. In FIG. 2, section 263 and/or section 266 of the column 261 comprise of theoretical separation stages in the form of structured, random packings or distillative trays that are commonly used in liquid-vapor distillation. The column hydraulics is set by the bottoms reboiler with adequate boil-up rate to provide the necessary vapor up-flow traffic through the column, and the overhead condenser provides sufficient liquid down-flow through the reflux rate. The effective vapor and liquid flow traffic is shown by 275 which keeps the column from either drying or flooding. Those skilled in the art will know how to adjust the reboiler boil-up and condenser reflux for achieving stable column hydraulics.

A stationary bed, containing solid acidic resin catalyst pellets, is mounted in the mid-section 245 of the column. The catalyst bed is held in place by similar arrangements as catalytic structured packings (such as Katapak-S) that are typically used in reactive distillation. There are more than one theoretical separation stages between the catalyst bed and overhead condenser and more than one theoretical stage between the catalyst bed and bottoms reboiler. The feed entry point can be above, middle or below the catalyst bed location. In this example, the liquid-feed enters above the catalyst bed via stream 215. The catalyst reaction zone 245 conditions are maintained such that the reaction of BDO to THF proceeds with high conversion and desired product selectivity. As the reaction continues the by-product water and THF product are driven out of the reaction zone as vapor. The vapor mixture (i.e., stream 220) is condensed in the overhead condenser and recovered as 80±20:20±20% THF:water distillate mixture. The THF-rich liquid distillate is drawn out of the system and processed downstream.

Fresh BDO is charged via stream 215 at a steady feed rate to replenish the converted feed. At steady state, the overhead product mass flow rate is roughly matched with the fresh BDO addition through stream 215. The column base continues to boil BDO which provides the reactant vapor feed to the reaction zone 245.

The undesired tar byproduct is concentrated and removed in the column base where the maximum temperature is observed. The bottom liquid stream 230 composition comprises mainly BDO with trace amounts of THF, water and a few % by weight tars.

All patents, patent applications, test procedures, priority documents, articles, publications, manuals, and other documents cited herein are fully incorporated by reference to the extent such disclosure is not inconsistent with this invention and for all jurisdictions in which such incorporation is permitted.

When numerical lower limits and numerical upper limits are listed herein, ranges from any lower limit to any upper limit are contemplated.

While the illustrative embodiments of the invention have been described with particularity, it will be understood that various other modifications will be apparent to and may be readily made by those skilled in the art without departing from the spirit and scope of the invention. Accordingly, it is not intended that the scope of the claims hereof be limited to the examples and descriptions set forth herein but rather that the claims be construed as encompassing all the features of patentable novelty which reside in the present invention, including all features which would be treated as equivalents thereof by those skilled in the art to which the invention pertains.

What is claimed is:

1. An improved process for making tetrahydrofuran, the process comprising:
providing a reaction mixture comprising 1,4-butanediol to a first zone;
maintaining conditions of temperature and pressure in the first zone sufficient to produce a vapor-rich region comprising the reaction mixture;
providing a second zone in the vapor-rich region between the first zone and a vapor condenser, wherein the second zone comprises an acid catalyst;
maintaining conditions of temperature and pressure in the second zone sufficient to effect dehydration of 1,4-butanediol and ring closure to form a product comprising tetrahydrofuran; and
recovering the product.

2. The process of claim 1, wherein the acid catalyst is a solid catalyst.

3. The process of claim 2, wherein the solid catalyst is selected from the group consisting of a polymer-based acid resin, a mineral-based supported acid catalyst and combinations thereof.

4. The process of claim 2, wherein the solid catalyst is an acidic resin catalyst.

5. The process of claim 4, wherein the acidic resin catalyst has an acid equivalency of between 1 and 10.

6. The process of claim 1, wherein the temperature of the second zone is from 80 to 250° C.

7. The process of claim 1, wherein the product is comprised of 80±20/20±20, weight/weight, tetrahydrofuran/water.

8. The process of claim 6, wherein the temperature of the second zone is from 110 to 250° C.

9. The process of claim 1, wherein the conditions of the second zone include a temperature of from 80 to 250° C. and pressure of from 200 to 10,000 mbar absolute.

10. The process of claim 9, wherein the conditions of the second zone include a temperature of from 110 to 250° C. and pressure of from 950 to 8,000 mbar absolute.

11. The process of claim 1, wherein the conditions of the second zone include a pressure high enough to retain the reaction mixture in liquid phase.

12. The process of claim 1, wherein the conditions of the second zone include a pressure low enough to allow tetrahydrofuran to boil out.

13. The process of claim 1, wherein the second zone operates adiabatically.

14. The process of claim 1, wherein the acid catalyst is contained within a fixed bed.

15. The process of claim 1, wherein the acid catalyst is contained within a structured distillation column packing bed.

16. The process of claim 1, wherein the acid catalyst is slurried.

17. The process of claim 1, wherein the yield loss of butanediol to tar is less than 1 percent by weight.

18. The process of claim 17, wherein the tar formation is determined by mass totaling method.
